(19) United States
(12) Patent Application Publication
(10) Pub. No.: US 2025/0075155 A1
(43) Pub. Date: Mar. 6, 2025

(54) Use of Vanillyl Ethers to Modify Flavor of Distilled Spirits

(71) Applicant: Firmenich Incorporated, Plainsboro, NJ (US)

(72) Inventors: Joseph R. Fotsing, San Diego, CA (US); Sylvain Lauvaux, Mont-Saint-Guibert (BE)

(73) Assignee: Firmenich Incorporated, Plainsboro, NJ (US)

(21) Appl. No.: 18/293,221

(22) PCT Filed: Sep. 23, 2022
(86) PCT No.: PCT/US2022/044469

§ 371 Date: Jan. 29, 2024

Related U.S. Application Data
(60) Provisional application No. 63/248,603, filed on Sep. 27, 2021.

(51) Int. Cl. C12G 3/06; A23L 27/00; A23L 27/20

(52) U.S. Cl. CPC C12G 3/06; A23L 27/204; A23L 27/88

(57) ABSTRACT

The present disclosure generally relates to the use of certain vanillyl ethers to modify the flavor of distilled spirits. In some embodiments, the flavor modification relates to enhancing the perceived smoothness of a distilled spirit, enhancing the mouthfeel of a distilled spirit, reducing the astringency of a distilled spirit, reducing the perceived burning effect of a distilled spirit, reducing the perceived bitterness of a distilled spirit, or any combination thereof. The disclosure also provides related methods of modifying the flavor of a distilled spirit. comprising introducing certain vanillyl ethers to a distilled spirit. The disclosure also provides beverage compositions that comprise a distilled spirit and certain vanillyl ethers.

### TECHNICAL FIELD

The present disclosure generally relates to the use of certain vanillyl ethers to modify the flavor of distilled spirits. In some embodiments, the flavor modification relates to enhancing the perceived smoothness of a distilled spirit, enhancing the mouthfeel of a distilled spirit, reducing the astringency of a distilled spirit, reducing the perceived burning effect of a distilled spirit, reducing the perceived bitterness of a distilled spirit, or any combination thereof. The disclosure also provides related methods of modifying the flavor of a distilled spirit, comprising introducing certain vanillyl ethers to a distilled spirit. The disclosure also provides beverage compositions that comprise a distilled spirit and certain vanillyl ethers.

### DESCRIPTION OF RELATED ART

Distilled spirits are widely consumed throughout the world. The initial stage in the production of such beverages involves fermenting a sugar-containing composition, typically at an alcohol concentration of no more than about 20% ethanol by volume, and often less. The sugar-containing composition can be derived from a variety of different sources. Typically, the sugar-containing composition is derived from a fruit, such as grapes, apples, pears, plums, and the like, or from a grain, such as barley, wheat, buckwheat, corn, rye, and the like, or from other sugar-containing plants, such as sugar cane, sugar beet, potatoes, yams, sweet potatoes, agave, and the like. In some cases, the process can involve some amount of pre-treatment of the fruit or grain. For example, in the case of many scotch whiskeys, the malted barley is heated in peat-fired ovens to stop the germination process. This transfers various phenolic compounds from the peat smoke into the grain, and gives certain scotches their distinctively peaty character. The sugar-containing composition is fermented into an ethanol-containing composition, generally with the assistance of various yeast strains. The resulting ethanol-containing composition typically has an ethanol content ranging from about 3% by volume up to about 20% by volume, depending on the density of sugars in the sugar source, and the tolerance of the yeast for higher concentrations of ethanol.

The distillation process is a concentration process. Because ethanol boils at a lower temperature than water (78° C. versus 100° C. under typical conditions), one can boil off the ethanol from the water by heating the ethanol-containing composition to a temperature between their two boiling points. A wide variety of different distillation apparatuses have been designed for this purpose. The resulting distillate contains a much higher concentration of ethanol, sometimes as high as about 95% ethanol by volume. Because the composition subjected to distillation is derived from certain natural products, a variety of other compounds are present in the ethanol-containing composition. Some of these may be present in the plant, while others are generated from such compounds by the yeast used to ferment the sugar to ethanol. Many of these compounds have boiling points near that of ethanol, and therefore pass over into the distillate during the distillation process. In some cases, these compounds can affect the taste of the spirit positively. But, in other cases, they have a negative effect on taste. Certain unpleasant tastes can be mitigated by multiple rounds of distillation, or by aging the spirit in wooden casks or barrels. But these processes can be costly, and drive the price of the spirit to a point where it becomes unaffordable to the average consumer. Further, the ethanol in the spirit can be perceived as bitter to some consumers

Therefore, there is a continuing need to develop more cost-effective ways at reducing the perceived astringency, bitterness, or sharpness of distilled spirits, so as to produce a spirit that tastes more like an aged or highly distilled spirit.

### SUMMARY

The present disclosure relates to the discovery that certain vanillyl ether derivatives, when added in small quantities to distilled spirits, can reduce certain off tastes and produce a spirit that exhibits a character more consistent with that of an aged spirit.

In a first aspect, the disclosure provides uses of vanillyl ether compounds to modify the flavor of a distilled spirit, wherein the vanillyl ether compounds are compounds of formula (I):

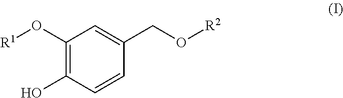

wherein:
R¹ is methyl or ethyl; and
R² is C₁₋₈ alkyl or C₃₋₈ cycloalkyl.
   In a related aspect, the disclosure provides methods of modifying the flavor of a distilled spirit, the method comprising introducing vanillyl ether compounds to a distilled spirit or to a beverage composition comprising a distilled spirit, wherein the vanillyl ether compounds are compounds of formula (I), as defined above. In some embodiments of the foregoing aspects, modifying the flavor comprises one of more of the following: reducing the astringency, reducing or masking the bitterness, reducing the burning, reducing the perceived sharpness, improving the mouthfeel, and improving the perceived sweetness. In some embodiments, the vanillyl ethers are introduced or used in the distilled spirit at a concentration ranging from 0.1 ppm to 100 ppm, or from 1 ppm to 50 ppm, based on the total weight of the distilled spirit. In a second aspect, the disclosure a beverage composition comprising a distilled ethanol composition and a vanillyl ether compound, wherein the vanillyl ether compound is a compound of formula (I):

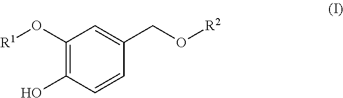

wherein:
R¹ is methyl or ethyl; and
R² is a hydrogen atom, C₁₋₈ alkyl, or C₃₋₈ cycloalkyl.
   In some embodiments, the vanillyl ethers are introduced or used in the distilled spirit at a concentration ranging from 0.1 ppm to 100 ppm, or from 1 ppm to 50 ppm, based on the total weight of the distilled spirit. In some embodiments, the beverage composition comprises at least 20% by volume ethanol.

Further aspects, and embodiments thereof, are set forth below in the Detailed Description, the Drawings, the Abstract, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows a chemical formula for vanillyl ether compounds whose use is described herein, wherein R¹ is methyl or ethyl, and R² is a hydrogen atom, alkyl, or cycloalkyl.

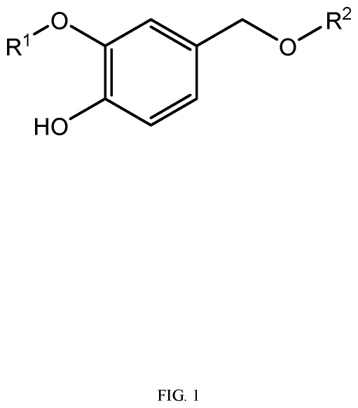

### DETAILED DESCRIPTION

The following Detailed Description sets forth various aspects and embodiments provided herein. The description is to be read from the perspective of the person of ordinary skill in the relevant art. Therefore, information that is well known to such ordinarily skilled artisans is not necessarily included.

### Definitions

The following terms and phrases have the meanings indicated below, unless otherwise provided herein. This disclosure may employ other terms and phrases not expressly defined herein. Such other terms and phrases have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, “Cₐ to C_{b}” or “C_{a-b}” in which “a” and “b” are integers, refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, —CH₃, —CH₂CH₃, —CH₂CH₂CH₃, —CH(CH₃)₂, —CH₂CH₂CH₂CH₃, —CH(CH₃)CH₂CH₃, and —C(CH₃)₃.

As used herein, “alkyl” means a straight or branched hydrocarbon chain that is fully saturated (i.e., contains no double or triple bonds). In some embodiments, an alkyl group has 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like. Unless indicated to the contrary, the term “alkyl” refers to a group that is not further substituted.

As used herein, “cycloalkyl” means a non-aromatic, saturated cyclic ring or ring system containing only carbon atoms in the ring system backbone. The cycloalkyl group may be designated as “C₃₋₈ cycloalkyl” or similar designations. Examples of cycloalkyl rings include, but are not limited to, cyclopropyl, cyclobutyl, cyclopentyl, cyclohexyl, cyclohexenyl, adamantyl, and spiro[4.4]nonanyl. Unless indicated to the contrary, the term “cycloalkyl” refers to a group that is not further substituted.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

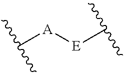

includes the substituent being oriented such that the A is attached at the leftmost attachment point of the molecule as well as the case in which A is attached at the rightmost attachment point of the molecule.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein, “comprise” or “comprises” or “comprising” or “comprised of” refer to groups that are open, meaning that the group can include additional members in addition to those expressly recited. For example, the phrase, “comprises A” means that A must be present, but that other members can be present too. The terms “include,” “have,” and “composed of” and their grammatical variants have the same meaning. In contrast, “consist of” or “consists of” or “consisting of” refer to groups that are closed. For example, the phrase “consists of A” means that A and only A is present.

As used herein, “optionally” means that the subsequently described event(s) may or may not occur. In some embodiments, the optional event does not occur. In some other embodiments, the optional event does occur one or more times.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, the term “distilled spirit” refers to an ethanol-containing composition formed by distilling the product of fermenting a sugar-containing composition derived from a natural source, such as fruit, grains, agave, sugar cane, and the like. Any suitable distillation process can be used. The distilled spirit can also be aged, for example, in oak barrels.

Other terms are defined in other portions of this description, even though not included in this subsection.

### Vanillyl Ether Compounds

The uses, methods, compositions, and products disclosed herein relate to vanillyl ether compounds, mainly in the context of ethanol-containing beverage compositions. The vanillyl ether compounds are compounds of formula (I):

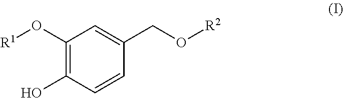

wherein:
R¹ is methyl or ethyl; and
R² is a hydrogen atom, C₁₋₈ alkyl, or C₃₋₈ cycloalkyl; or R² is —CH₂-(phenyl), wherein the phenyl moiety is optionally substituted one or two times by substituents selected independently from the group consisting of —OH and —OCH₃.

The variable R¹ can have any suitable value, according to the definition set forth above. In some embodiments, R¹ is methyl. In some embodiments, R¹ is ethyl.

The variable R² can have any suitable value, according to the definition set forth above. In some embodiments, R² is C₁₋₈ alkyl. In some such embodiments, R²is methyl, ethyl, propyl, butyl, pentyl, hexyl, isopropyl, butan-2-yl, pentan-2-yl, hexan-2-yl, isobutyl, 2-methylbutyl, isopentyl, or pentan-3-yl. In some embodiments, R² is ethyl or butyl. In some embodiments, R² is ethyl. In some other embodiments, R² is C₃₋₈ cycloalkyl, such as cyclopentyl, cyclohexyl, cycloheptyl, or cyclooctyl. In some embodiments, R² is cyclopentyl or cyclohexyl. In some other embodiments, R² is a hydrogen atom. In some other embodiments, R² is —CH₂-(phenyl), wherein the phenyl moiety is optionally substituted one or two times by substituents selected independently from the group consisting of —OH and —OCH₃. In some such embodiments, R² is benzyl. In some other such embodiments, R² is —CH₂-(phenyl), wherein the phenyl moiety is substituted at the 3-position or 4-position by at least one or two substituents selected independently from the group consisting of —OH and —OCH₃.

Table 1 below sets forth various examples of vanillyl ether compounds according to certain embodiments. Each compound is itself a separate embodiment of a vanillyl ether compound.

TABLE 1

| No. | Compound |
|---|---|
| 101 | 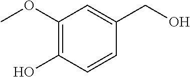 |
| | 4-(hydroxymethyl)-2-methoxyphenol |
| 102 | 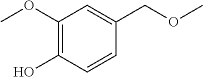 |
| | 2-methoxy-4-(methoxymethyl)phenol |
| 103 | 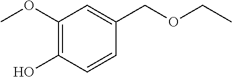 |
| | 4-(ethoxymethyl)-2-methoxyphenol (VEE) |
| 104 | 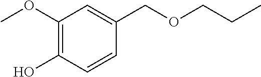 |
| | 2-methoxy-4-(propoxymethyl)phenol |
| 105 | 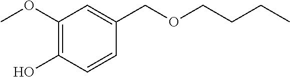 |
| | 4-(butoxymethyl)-2-methoxyphenol (VBE) |
| 106 | 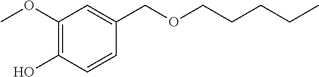 [SMILES: CCCCCOCc1ccc(O)c(OC)c1] |
| | 2-methoxy-4-((pentyloxy)methyl)phenol |
| 107 | 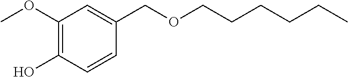 [SMILES: CCCCCCOCc1ccc(O)c(OC)c1] |
| | 4-((hexyloxy)methyl)-2-methoxyphenol |
| 108 | 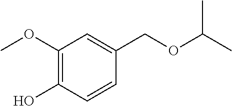 |
| | 4-(isopropoxymethyl)-2-methoxyphenol |
| 109 | 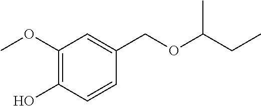 |
| | 4-(sec-butoxymethyl)-2-methoxyphenol |
| 110 | 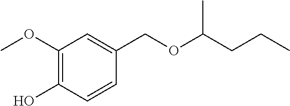 [SMILES: CCCC(C)OCc1ccc(O)c(OC)c1] |
| | 2-methoxy-4-((pentan-2-yloxy)methyl)phenol |
| 111 | 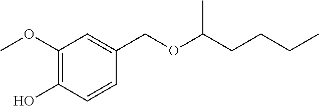 [SMILES: CCCCC(C)OCc1ccc(O)c(OC)c1] |
| | 4-((hexan-2-yloxy)methyl)-2-methoxyphenol |
| 112 | 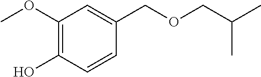 |
| | 4-(isobutoxymethyl)-2-methoxyphenol |
| 113 | 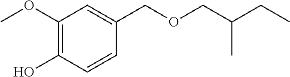 [SMILES: CCC(C)COCc1ccc(O)c(OC)c1] |
| | 2-methoxy-4-((2-methylbutoxy)methyl)phenol |
| 114 | 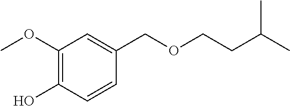 [SMILES: COc1cc(COCCC(C)C)ccc1O] |
| | 4-((isopentyloxy)methyl)-2-methoxyphenol |
| 115 | 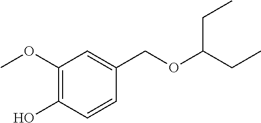 [SMILES: CCC(CC)OCc1ccc(O)c(OC)c1] |
| | 2-methoxy-4-((pentan-3-yloxy)methyl)phenol |
| 116 | 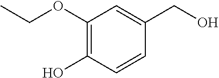 |
| | 2-ethoxy-4-(hydroxymethyl)phenol |
| 117 | 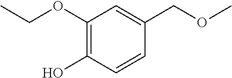 |
| | 2-ethoxy-4-(methoxymethyl)phenol |
| 118 | 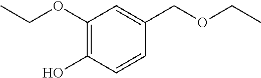 |
| | 2-ethoxy-4-(ethoxymethyl)phenol |
| 119 | 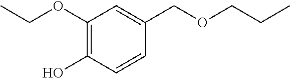 |
| | 2-ethoxy-4-(propoxymethyl)phenol |
| 120 | 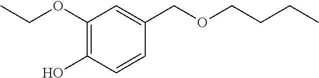 [SMILES: CCCCOCc1ccc(O)c(OCC)c1] |
| | 4-(butoxymethyl)-2-ethoxyphenol |
| 121 | 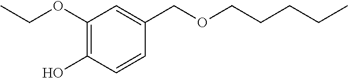 [SMILES: CCCCCOCc1ccc(O)c(OCC)c1] |
| | 2-ethoxy-4-((pentyloxy)methyl)phenol |
| 122 | 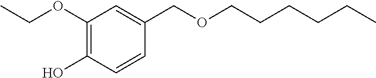 [SMILES: CCCCCCOCc1ccc(O)c(OCC)c1] |
| | 2-ethoxy-4-((hexyloxy)methyl)phenol |
| 123 | 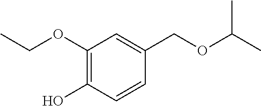 |
| | 2-ethoxy-4-(isopropoxymethyl)phenol |
| 124 | 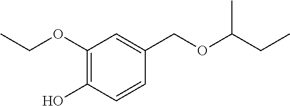 [SMILES: CCOc1cc(COC(C)CC)ccc1O] |
| | 4-(sec-butoxymethyl)-2-ethoxyphenol |
| 125 | 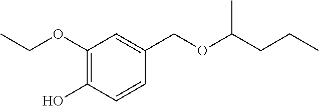 [SMILES: CCCC(C)OCc1ccc(O)c(OCC)c1] |
| | 2-ethoxy-4-((pentan-2-yloxy)methyl)phenol |
| 126 | 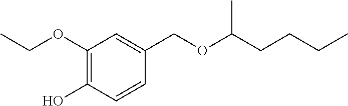 [SMILES: CCCCC(C)OCc1ccc(O)c(OCC)c1] |
| | 2-ethoxy-4-((hexan-2-yloxy)methyl)phenol |
| 127 | 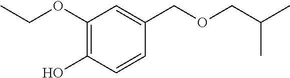 [SMILES: CCOc1cc(COCC(C)C)ccc1O] |
| | 2-ethoxy-4-(isobutoxymethyl)phenol |
| 128 | 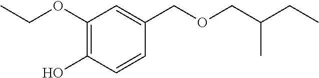 [SMILES: CCOc1cc(COCC(C)CC)ccc1O] |
| | 2-ethoxy-4-((2-methylbutoxy)methyl)phenol |
| 129 | 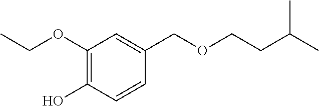 [SMILES: CCOc1cc(COCCC(C)C)ccc1O] |
| | 2-ethoxy-4-((isopentyloxy)methyl)phenol |
| 130 | 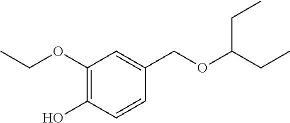 [SMILES: CCOc1cc(COC(CC)CC)ccc1O] |
| | 2-ethoxy-4-((pentan-3-yloxy)methyl)phenol |
| 131 | 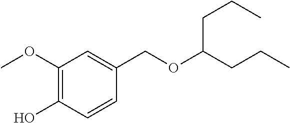 [SMILES: CCCC(CCC)OCc1ccc(O)c(OC)c1] |
| | 4-((heptan-4-yloxy)methyl)-2-methoxyphenol |
| 132 | 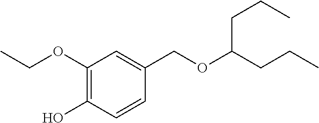 [SMILES: CCCC(CCC)OCc1ccc(O)c(OCC)c1] |
| | 2-ethoxy-4-((heptan-4-yloxy)methyl)phenol |
| 133 | 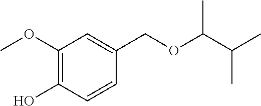 [SMILES: COc1cc(COC(C)C(C)C)ccc1O] |
| | 2-methoxy-4-(((3-methylbutan-2-y1)oxy)methyl)phenol |
| 134 | 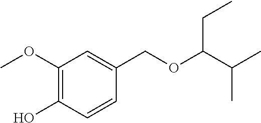 [SMILES: CCC(OCc1ccc(O)c(OC)c1)C(C)C] |
| | 2-methoxy-4-(((2-methylpentan-3-yl)oxy)methyl)phenol |
| 135 | 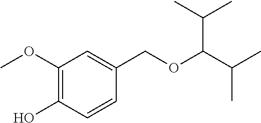 [SMILES: COc1cc(COC(C(C)C)C(C)C)ccc1O] |
| | 4-(((2,4-dimethylpentan-3-y1)oxy)methyl)- |
| | 2-methoxyphenol |
| 136 | 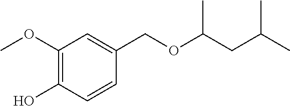 [SMILES: COc1cc(COC(C)CC(C)C)ccc1O] |
| | 2-methoxy-4-(((4-methylpentan-2-yl)oxy)methyl)phenol |
| 137 | 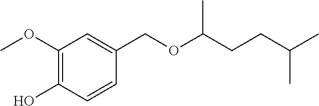 [SMILES: COc1cc(COC(C)CCC(C)C)ccc1O] |
| | 2-methoxy-4-(((5-methylhexan-2-yl)oxy)methyl)phenol |
| 138 | 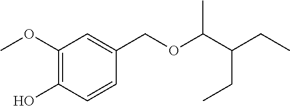 [SMILES: CCC(CC)C(C)OCc1ccc(O)c(OC)c1] |
| | 4-(((3-ethylpentan-2-yl)oxy)methyl)-2-methoxyphenol |
| 139 | 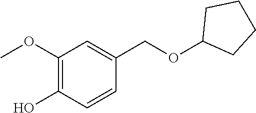 [SMILES: COc1cc(COC2CCCC2)ccc1O] |
| | 4-((cyclopentyloxy)methyl)-2-methoxyphenol |
| 140 | 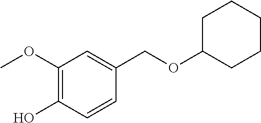 [SMILES: COc1cc(COC2CCCCC2)ccc1O] |
| | 4-((cyclohexyloxy)methyl)-2-methoxyphenol |
| 141 | 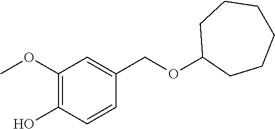 [SMILES: COc1cc(COC2CCCCCC2)ccc1O] |
| | 4-((cycloheptyloxy)methyl)-2-methoxyphenol |
| 142 | 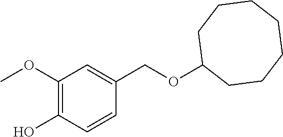 [SMILES: COc1cc(COC2CCCCCCC2)ccc1O] |
| | 4-((cyclooctyloxy)methyl)-2-methoxyphenol |
| 143 | 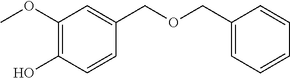 [SMILES: COc1cc(COCc2ccccc2)ccc1O] |
| | 4-((benzyloxy)methyl)-2-methoxyphenol |
| 144 | 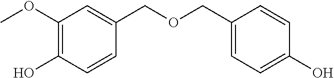 [SMILES: COc1cc(COCc2ccc(O)cc2)ccc1O] |
| | 4-(((4-hydroxybenzyl)oxy)methyl)-2-methoxyphenol |
| 145 | 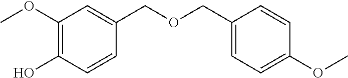 |
| | 2-methoxy-4-(((4-methoxybenzyl)oxy)methyl)phenol |
| 146 | 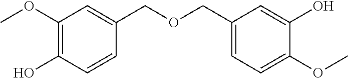 |
| | 4-(((3-hydroxy-4-methoxybenzyl)oxy)methyl)- |
| | 2-methoxyphenol |
| 147 | 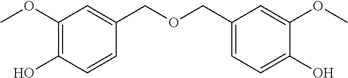 |
| | 4,4′-(oxybis(methylene))bis(2-methoxyphenol) |
| 148 | 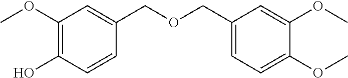 |
| | 4-(((3,4-dimethoxybenzyl)oxy)methyl)- |
| | 2-methoxyphenol |

Isotopes may be present in any of the vanillyl ether compounds described. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

In some embodiments, the vanillyl ether compounds capable of forming acid or base salts by virtue of the presence of amino or carboxyl groups or groups similar thereto. Comestibly acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Comestibly acceptable salts can be formed using inorganic and organic bases. Inorganic bases from which salts can be derived include, for example, bases that contain sodium, potassium, lithium, ammonium, calcium, magnesium, iron, zinc, copper, manganese, aluminum, and the like; particularly preferred are the ammonium, potassium, sodium, calcium and magnesium salts. In some embodiments, treatment of the vanillyl ether compounds disclosed herein with an inorganic base results in loss of a labile hydrogen from the compound to afford the salt form including an inorganic cation such as Li⁺, Na⁺, K⁺, Mg²⁺ and Ca²⁺ and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the salts are comestibly acceptable salts, which are salts suitable for inclusion in ingestible compositions, such as food or beverage products.

In some embodiments, the vanillyl ether compound is in its free (non-salt) form. In other embodiments, the vanillyl ether compound is a comestibly acceptable salt of any of the compounds set forth above.

In some embodiments, the vanillyl ether compounds exist as a crystalline solid, either in substantially pure form or in a formulation such as those set forth below. The crystalline solid can have any suitable polymorphic form, such as any polymorphic form obtainable via recrystallization in any suitable solvent system, according to techniques commonly used in the art of polymorph screening.

In some other embodiments, the vanillyl ether compounds exist as an amorphous solid or a semi-amorphous solid, meaning that it lacks any regular crystalline structure. Such solids can be generated using standard techniques, such as spray drying, and the like.

In some embodiments, the vanillyl ether compounds exist as a solvate, which is a pseudomorphic form of the compound in which one or more solvent molecules (such as water molecules) are taken up into the crystalline structure. Any suitable solvent or combination of solvents can be used, including, but not limited to, water, methanol, ethanol, n-propanol, isopropanol, n-butanol, 2-butanol, isobutanol, ethyl acetate, ethylene glycol, 1,2-propylene glycol, 1,3-propylene glycol, and the like. In some embodiments, the disclosure provides hydrates of the vanillyl ether compounds. Such solvates can be generated by any suitable means, such as those techniques typically used by skilled artisans in the field of polymorph and solvate screening.

In some other embodiments, the vanillyl ether compounds exist as a co-crystal with one or more other compounds, such as one or more other sweetener compounds. The vanillyl ether compounds can form a co-crystal with any suitable compound. Non-limiting examples of such suitable compounds include fructose, glucose, galactose, sucrose, lactose, maltose, allulose, sugar alcohols (such as erythritol, sorbitol, xylitol, and the like), sucralose, steviol glycosides (such as rebaudioside A, rebaudioside B, rebaudioside D, rebaudioside E, rebaudioside M, and the like natural stevioside compounds), other mogrosides (such as mogroside V, isomogroside V, siamenoside I, isomogroside III_{E}, the 1,6-α isomer of siamenoside I, and the like), aspartame, saccharin, acesulfame K, cyclamate, inulin, isomalt, and maltitol. Such co-crystals can be generated by any suitable means, such as those set forth in U.S. Patent Application Publication No. 2018/0363074, which is incorporated herein by reference.

In some embodiments, the vanillyl ether compounds are in the form of a dry particle. Such dry particles can be formed by standard techniques in the art, such as dry granulation, wet granulation, and the like. Such particles can also contain a number of excipients, including, inert diluents, such as calcium carbonate, sodium carbonate, lactose, calcium phosphate, and sodium phosphate; granulating and disintegrating agents, such as starch, cellulosic materials, and alginic acid; binding agents, such as gelatin, guar gum, and acacia; and lubricating agents, such as magnesium stearate, stearic acid, and talc. Other excipients typically used in food and beverage products can also be included, such as typical foodstuff materials.

In some embodiments, the vanillyl ether compounds are in the form of a liquid solution or a liquid suspension. Such compositions can also include: carboxymethylcellulose, methylcellulose, hydroxy propylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Such compositions can also include one or more coloring agents, one or more flavoring agents, and the like. Such liquid suspensions and solutions have a liquid carrier. In general, the liquid carrier comprises water. In some such cases, the liquid composition is an emulsion, such as an oil-in-water or a water-in-oil emulsion. Further, in some cases, water may be too polar to dissolve the vanillyl ether compounds to the desired concentration. In such instances, it can be desirable to introduce water-miscible solvents, such as alcohols, glycols, polyols, and the like, to the solvent to enhance solubilization of the vanillyl ether compounds.

In some embodiments, the vanillyl ether compounds are in the form of a solution, i.e., are solvated within a liquid carrier. In some embodiments, the liquid carrier is an aqueous carrier. Such solutions can be diluted to any suitable concentration.

### Uses and Methods

In certain aspects, the disclosure provides uses of vanillyl ether compounds according to any of the embodiments set forth above to modify the flavor of a distilled spirit. In a related aspect, the disclosure provides methods of modifying the flavor of a distilled spirit, the method comprising introducing vanillyl ether compounds to a distilled spirit or to a beverage composition comprising a distilled spirit.

In general, the use of the vanillyl ether compounds in combination with a distilled spirit improves the taste of the distilled spirit, or a beverage made therefrom, by giving the spirit or beverage a more rounded and less sharp taste. This is especially true where the distilled spirit is grain alcohol, which is often blended with more refined spirits to reduce the cost of the overall beverage. The vanillyl ether compounds can reduce some of the unpleasant taste of the grain alcohol, and thereby make a cheaper beverage taste like a more expensive beverage. In other instances, including the vanillyl ether compounds can reduce the number of times that a spirit needs to be distilled.

In one embodiment of the uses and methods set forth herein, modifying the flavor of the distilled spirit comprises reducing the astringency of the distilled spirit. In another embodiment of the uses and methods set forth herein, modifying the flavor of the distilled spirit comprises reducing or masking the bitterness of the distilled spirit. In another embodiment of the uses and methods set forth herein, modifying the flavor of the distilled spirit comprises reducing the burning of the distilled spirit. In another embodiment of the uses and methods set forth herein, modifying the flavor of the distilled spirit comprises reducing the sharpness of the distilled spirit. In another embodiment of the uses and methods set forth herein, modifying the flavor of the distilled spirit comprises enhancing the mouthfeel of the distilled spirit. In another embodiment of the uses and methods set forth herein, modifying the flavor of the distilled spirit comprises enhancing the perceived roundedness of the distilled spirit. In another embodiment of the uses and methods set forth herein, modifying the flavor of the distilled spirit comprises enhancing the perceived sweetness of the distilled spirit.

The vanillyl ether compounds can be used at any suitable concentration in the distilled spirit or the resulting beverage. In some embodiments, the vanillyl ethers are introduced or used in the distilled spirit or beverage at a concentration ranging from 0.01 ppm to 100 ppm, 0.1 ppm to 100 ppm, or from 1 ppm to 50 ppm, or from 2 ppm to 25 ppm, based on the total weight of the distilled spirit or beverage.

Any suitable distilled spirit can be used. In some embodiments, the distilled spirit is derived from fermenting a composition comprising the juice of one of more fruits. Some non-limiting examples of fruit that can be used include grapes, apples, peaches, nectarines, pears, and the like. In some other embodiments, the distilled spirit is derived from fermenting a composition comprising enzyme-treated grain products in which the starch in the grain has been converted to fermentable sugars. Some non-limiting examples of grains that can be used include barley, wheat, corn, rye, rice, potato, yam, sweet potato, and the like. Such grain-derived distilled spirits can be referred to as “grain spirits.” In some embodiments, the distilled spirit is a grain spirit. Other sugar-containing materials, such as sugar cane, sugar beet, and agave, can also be used.

The distilled spirits always contain some amount of water. In some cases, where the distillation is particularly efficient, the distilled spirit comprises only about 5% water by volume. In general, the percentage is much higher. In some embodiments, the distilled spirit is diluted via the addition of water following the distillation.

In general, the distilled spirit contains “congeners,” which are taste or aroma compounds that pass from the fermented product into the distilled spirit during the distillation process. Such compounds provide various distilled spirits with their characteristic taste and aroma profile. In some cases, these congeners impart pleasant tasted. For example, beverages such as brandy and grappa can exhibit a characteristically sweet taste due to the presence of certain congeners that impart a perceived sweetness. But other congeners impart an unpleasant taste, such as the sharp or astringent taste characteristic of unaged grain spirit. Distilled spirits are often aged in wooden casks or barrels for the purpose of reducing the presence of such unpleasant congeners (for example, via chemical reactions or evaporation) or to introduce compounds from the cask or barrel that mask the unpleasant taste of certain unpleasant congeners.

As used herein, the term “distilled spirit” can refer to either unaged or aged spirits. In some embodiments, the distilled spirit is an unaged distilled spirit, meaning that the spirit has not been aged for any time in a wooden cask or barrel. In some other embodiments, the distilled spirit is an aged spirit, which has been aged in a wooden cask or barrel for a period of time, for example, no more than 1 year, or no more than 2 years, or no more than 3 years, or no more than 4 years, or no more than 5 years, or no more than 6 years.

The preceding uses and methods involve using the using or introducing the vanillyl ether compounds with or to a distilled spirit. Those distilled spirits plus the vanillyl ether compounds can themselves form beverage compositions, for example, with some dilution with water to reduce the alcohol content of the distilled spirit to a more palatable level, such as at least 20% alcohol by volume, or at least 25% alcohol by volume, or at least 30% alcohol by volume, or at least 35% alcohol by volume, or at least 40% alcohol by volume. Such beverage compositions may contain other ingredients, however, as described below.

### Beverage Compositions

In certain aspects, the disclosure provides beverage compositions comprising a distilled spirit and a vanillyl ether compound.

Any suitable distilled spirit can be used. In some embodiments, the distilled spirit is derived from fermenting a composition comprising the juice of one of more fruits. Some non-limiting examples of fruit that can be used include grapes, apples, peaches, nectarines, pears, and the like. In some other embodiments, the distilled spirit is derived from fermenting a composition comprising enzyme-treated grain products in which the starch in the grain has been converted to fermentable sugars. Some non-limiting examples of grains that can be used include barley, wheat, corn, rye, rice, potato, yam, sweet potato, and the like. Such grain-derived distilled spirits can be referred to as “grain spirits.” In some embodiments, the distilled spirit is a grain spirit. Other sugar-containing materials, such as sugar cane, sugar beet, and agave, can also be used.

The distilled spirits always contain some amount of water. In some cases, where the distillation is particularly efficient, the distilled spirit comprises only about 5% water by volume. In general, the percentage is much higher. In some embodiments, the distilled spirit is diluted via the addition of water following the distillation.

In general, the distilled spirit contains “congeners,” which are taste or aroma compounds that pass from the fermented product into the distilled spirit during the distillation process. Such compounds provide various distilled spirits with their characteristic taste and aroma profile. In some cases, these congeners impart pleasant tasted. For example, beverages such as brandy and grappa can exhibit a characteristically sweet taste due to the presence of certain congeners that impart a perceived sweetness. But other congeners impart an unpleasant taste, such as the sharp or astringent taste characteristic of unaged grain spirit. Distilled spirits are often aged in wooden casks or barrels for the purpose of reducing the presence of such unpleasant congeners (for example, via chemical reactions or evaporation) or to introduce compounds from the cask or barrel that mask the unpleasant taste of certain unpleasant congeners.

As used herein, the term “distilled spirit” can refer to either unaged or aged spirits. In some embodiments, the distilled spirit is an unaged distilled spirit, meaning that the spirit has not been aged for any time in a wooden cask or barrel. In some other embodiments, the distilled spirit is an aged spirit, which has been aged in a wooden cask or barrel for a period of time, for example, no more than 1 year, or no more than 2 years, or no more than 3 years, or no more than 4 years, or no more than 5 years, or no more than 6 years.

In some embodiments, the distilled spirit can be diluted with water to reduce the alcohol content of the distilled spirit to a more palatable level, such as at least 20% alcohol by volume, or at least 25% alcohol by volume, or at least 30% alcohol by volume, or at least 35% alcohol by volume, or at least 40% alcohol by volume.

In some embodiments, the beverage composition comprises a blend of distilled spirits. For example, the beverage composition can comprise a blend of an unaged distilled spirit with one or more aged distilled spirits, such that the inclusion of the vanillyl ether compound in the beverage composition allows one to use the unaged distilled spirit at higher concentrations without imparting an unpleasant taste.

The beverage composition can include the vanillyl ether compound at any suitable concentration. For example, in some embodiments, the beverage composition comprises the vanillyl ether compound at a concentration ranging from 0.01 ppm to 100 ppm, 0.1 ppm to 100 ppm, or from 1 ppm to 50 ppm, or from 2 ppm to 25 ppm, based on the total weight the beverage composition.

In some embodiments, the beverage composition comprises one or more bitter tastants. In some embodiments, the bitter tastant is a high-intensity sweetener, such as acesulfame potassium, aspartame, neotame, cyclamate, saccharin, sucralose, steviol glycodises (such as rebaudioside A, rebaudioside B, rebaudioside M, rebaudioside D, or rebaudioside E), and mogrosides (such as mogroside III, mogroside IV, mogroside V, siamenoside I, isomogroside V, mogroside IV_{E}, isomogroside IV, mogroside III_{E}, 11-oxomogroside V, or the 1,6-α isomer of siamenoside I).

In some embodiments, the bitter tastant is a potassium salt, such as potassium chloride, which is often used as a partial or complete replacement of sodium chloride in certain low-sodium or zero-sodium beverages.

In some embodiments, the bitter tastant is caffeine, quinine, green tea, catechins, polyphenols (such as a polyphenol antioxidants), tannins, green robusta coffee extract, green coffee extract, menthol, and the like. Such compounds commonly occur in various natural foods products, such as tea and coffee, and in packaged food products, such as instant tea, instant coffee, packaged beverages, and the like.

The beverage composition can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose, D-psicose, L-ribose, D-tagatose, L-glucose, L-fucose, L-arbinose, D-turanose, and D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, allulose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A, other sweet Stevia-based glycosides, chemically modified steviol glycosides (such as glucosylated steviol glycosides), mogrosides, chemically modified mogrosides (such as glucosylated mogrosides), carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green stevia powder, stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, glycyrrhetic acid monoglucuronide, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, siraitia grosvenorii, soy bean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of stevia-derived sweeteners, such as steviol glycosides, glucosylated steviol glycosides, or rebaudiosides.

The beverage compositions can, in certain embodiments, comprise any additional ingredients or combination of ingredients as are commonly used in beverage products, including, but not limited to:
acids, including, for example citric acid, phosphoric acid, ascorbic acid, sodium acid sulfate, lactic acid, or tartaric acid;
coloring agents, including, for example caramel color, Red #40, Yellow #5, Yellow #6, Blue #1, Red #3, purple carrot, black carrot juice, purple sweet potato, vegetable juice, fruit juice, beta carotene, turmeric curcumin, or titanium dioxide;
preservatives, including, for example sodium benzoate, potassium benzoate, potassium sorbate, sodium metabisulfate, sorbic acid, or benzoic acid;
antioxidants including, for example ascorbic acid, calcium disodium EDTA, alpha tocopherols, mixed tocopherols, rosemary extract, grape seed extract, resveratrol, or sodium hexametaphosphate;
vitamins or functional ingredients including, for example resveratrol, Co-Q10, omega 3 fatty acids, theanine, choline chloride (citocoline), fibersol, inulin (chicory root), taurine, panax ginseng extract, guanana extract, ginger extract, L-phenylalanine, L-carnitine, L-tartrate, D-glucoronolactone, inositol, bioflavonoids, Echinacea, ginko biloba, yerba mate, flax seed oil, garcinia cambogia rind extract, white tea extract, ribose, milk thistle extract, grape seed extract, pyrodixine HCl (vitamin B6), cyanoobalamin (vitamin B12), niacinamide (vitamin B3), biotin, calcium lactate, calcium pantothenate (pantothenic acid), calcium phosphate, calcium carbonate, chromium chloride, chromium polynicotinate, cupric sulfate, folic acid, ferric pyrophosphate, iron, magnesium lactate, magnesium carbonate, magnesium sulfate, monopotassium phosphate, monosodium phosphate, phosphorus, potassium iodide, potassium phosphate, riboflavin, sodium sulfate, sodium gluconate, sodium polyphosphate, sodium bicarbonate, thiamine mononitrate, vitamin D3, vitamin A palmitate, zinc gluconate, zinc lactate, or zinc sulphate;
clouding agents, including, for example ester gun, brominated vegetable oil (BVO), or sucrose acetate isobutyrate (SAIB);
buffers, including, for example sodium citrate, potassium citrate, or salt;
flavors, including, for example propylene glycol, ethyl alcohol, glycerine, gum Arabic (gum acacia), maltodextrin, modified corn starch, dextrose, natural flavor, natural flavor with other natural flavors (natural flavor WONF), natural and artificial flavors, artificial flavor, silicon dioxide, magnesium carbonate, or tricalcium phosphate; or
starches and stabilizers, including, for example pectin, xanthan gum, carboxylmethylcellulose (CMC), polysorbate 60, polysorbate 80, medium chain triglycerides, cellulose gel, cellulose gum, sodium caseinate, modified food starch, gum Arabic (gum acacia), inulin, or carrageenan.

The beverage compositions can have any suitable pH. In some embodiments, the flavor-modifying compounds enhance the sweetness of a sweetener under a broad range of pH, e.g., from lower pH to neutral pH. The lower and neutral pH includes, but is not limited to, a pH from 1.5 to 9.0, or from 2.5 to 8.5; from 3.0 to 8.0; from 3.5 to 7.5; and from 4.0 to 7. In certain embodiments, compounds as disclosed and described herein, individually or in combination, can enhance the perceived sweetness of a fixed concentration of a sweetener in taste tests at a compound concentration of 50 μM, 40 μM, 30 μM, 20 μM, or 10 μM at both low to neutral pH value. In certain embodiments, the enhancement factor of the compounds as disclosed and described herein, individually or in combination, at the lower pH is substantially similar to the enhancement factor of the compounds at neutral pH. Such consistent sweet enhancing property under a broad range of pH allow a broad use in a wide variety of foods and beverages of the compounds as disclosed and described herein, individually or in combination.

In some embodiments, the beverage composition comprises a flavoring. Any suitable flavoring can be used. In some embodiments, the flavoring comprises synthetic flavor oils and flavoring aromatics or oils, oleoresins and extracts derived from plants, leaves, flowers, fruits, and so forth, or combinations thereof. Non-limiting examples of flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Non-limiting examples of other flavors include natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, watermelon, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a chamomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. In the context of dairy or dairy analog products, the most commonly used flavor agents are agents that impart flavors such as vanilla, French vanilla, chocolate, banana, lemon, hazelnut, coconut, almond, strawberry, mocha, coffee, tea, chai, cinnamon, caramel, cream, brown sugar, toffee, pecan, butter pecan, toffee, Irish crème, white chocolate, raspberry, pumpkin pie spice, peppermint, or any combination thereof.

In some embodiments, the beverage composition comprises vanillin or a vanillin analogue, which impart a vanilla flavor to the flavoring. In some further embodiments, the ingestible composition comprises one or more lactones, which impart a creamy flavor to the composition.

In some embodiments, the beverage composition comprises a sweetness enhancer. Any suitable sweetness enhancer can be used in the ingestible compositions disclosed herein, including synthetic sweetness enhancers, natural sweetness enhancers, or any combinations thereof.

Examples of suitable synthetic sweetness enhancers include, but are not limited to, N-(1-((4-amino-2,2-dioxo-1H-benzo[c][1,2,6]thiadiazin-5-yl)oxy)-2-methylpropan-2-yl)isonicotinamide, or any of its comestbly acceptable salts, 3-hydroxy benzoic acid, or any compounds set forth in U.S. Pat. Nos. 8,541,421; 8,815,956; 9,834,544; 8,592,592; 8,877,922; 9,000,054; and 9,000,051, as well as U.S. Patent Application Publication No. 2017/0119032.

Suitable examples of natural sweetness enhancers include, but are not limited to, hesperetin dihydrochalcone, hesperetin dihydrochalcone-4′-O'glucoside, neohesperetin dihydrochalcone, brazzein, hesperidin, phyllodulcin, naringenin, naringin, phloretin, glucosylated steviol glycosides, (2R,3R)-3-acetoxy-5,7,4′-trihydroxyflavanone, (2R,3R)-3-acetoxy-5,7,3 -trihydroxy-4′-methoxyflavanone, rubusosides, thaumatin, monellin, miraculin, glycyrrhizin and comestible acceptable salts thereof (such as the mono-ammonium salt), naringin dihydrochalcone, myricetin, nobiletin, polymethoxy flavones, mixed methoxy-and hydroxyflavones, quercetin, certain amino acids, and the like. As used herein, the term “glucosylated steviol glycoside” refers to the product of enzymatically glucosylating natural steviol glycoside compounds. The glucosylation generally occurs through a glycosidic bond, such as an α-1,2 bond, an α-1,4 bond, an α-1.6 bond, a β-1,2 bond, a β-1,4 bond, a β-1,6 bond, and so forth.

In some embodiments of any of the preceding embodiments, the beverage composition comprises 3-((4-amino-2,2-dioxo-1H-benzo[c][1,2,6]thiadiazin-5-yl)oxy)-2,2-dimethyl-N-propyl-propanamide, N-(1-((4-amino-2,2-dioxo-1H-benzo[c][1,2,6]-thiadiazin-5-yl)oxy)-2-methyl-propan-2-yl)isonicotinamide, or a comestibly acceptable salt thereof. In some embodiments, the flavor-modifying composition comprises N-(1-((4-amino-2,2-dioxo-1H-benzo[c][1,2,6]thiadiazin-5-yl)oxy)-2-methyl-propan-2-yl)isonicotinamide, or a comestbly acceptable salt thereof. In some embodiments, the flavor-modifying composition comprises N-(1-((4-amino-2,2-dioxo-1H-benzo[c][1,2,6]thiadiazin-5-yl)oxy)-2-methyl-propan-2-yl)isonicotinamide.

In some embodiments, the beverage composition comprises one or more umami enhancing compounds. Such umami enhancing compounds include, but are not limited to, naturally derived compounds, or synthetic compounds, such as any compounds set forth in U.S. Pat. Nos. 8,735,081; 8,124,121; and 8,968,708. In some embodiments, the umami-enhancing compound is (2R,4R)-1,2,4-trihydroxy-heptadec-16-ene, (2R,4R)-1,2,4-trihydroxyheptadec-16-yne, or a mixture thereof. In some embodiments, the umami-enhancing compound is (3R,5S)-1-(4-hydroxy-3-methoxyphenyl)decane-3,5-diol diacetate. In some embodiments, the umami-enhancing compound is N-(heptan-4-yl)benzo-[d][1,3]dioxole-5-carboxamide.

In some further embodiments, the beverage composition comprises one or more cooling enhancing compounds. Such cooling enhancing compounds include, but are not limited to, naturally derived compounds, such as menthol or analogs thereof, or synthetic compounds, such as any compounds set forth in U.S. Pat. Nos. 9,394,287 and 10,421,727. Non-limiting examples include N-ethyl-N-(thiophen-2-ylmethyl)-2-(p-tolyloxy)acetamide, N-(1H-pyrazol-3-yl)-N-(thiophen-2-ylmethyl)-2-(p-tolyloxy)acetamide, 2-(4-fluorophenoxy)-N-(1H-pyrazol-3-yl)-N-(thiophen-2-ylmethyl)acetamide, 2-(2-hydroxy-4-methylphenoxy)-N-(1H-pyrazol-3-yl)-N-(thiophen-2-ylmethyl)-acetamide, 2-((2,3-dihydro-1H-inden-5-yl)oxy)-N-(1H-pyrazol-3-yl)-N-(thiophen-2-ylmethyl)-acetamide, 2-((2,3-dihydro-1H-inden-5-yl)oxy)-N-(IH-pyrazol-3-yl)-N-(thiazol-5-ylmethyl)-acetamide, 2-((5-methoxy benzofuran-2-yl)oxy)-N-(1H-pyrazol-3-yl)-N-(thiophen-2-ylmethyl)-acetamide, (E/Z)-2-methyl-2-butenal, (E/Z)-2-isopropyl-5-methyl-2-hexenal, phloretin, naringenin, and any combinations thereof.

In some further embodiments, the beverage composition comprises one or more bitterness blocking or bitter masking compounds. Such bitterness blocking compounds or bitter masking include, but are not limited to, naturally derived compounds or synthetic compounds, such as any compounds set forth in U.S. Pat. Nos. 8,076,491; 8,445,692; and 9,247,759. Non-limiting examples include 3-(1-((3,5-dimethylisoxazol-4-yl)-methyl)-1H-pyrazol-4-yl)-1-(3-hydroxy benzyl)-imidazolidine-2,4-dione, 4-(2,2,3-trimethylcyclopentyl)butanoic acid, 3β-hydroxy dihydrocostunolide, 3β-hydroxypelenolide, probenecid, sakuranetin, 6-methoxysakuranetin, jaceosidin, 4′-fluoro-6-methoxyflavonone, 6,3 -dimethoxyflavonone, 6-methoxyflavonone, γ-aminobutyric acid, Nα,Nα-bis(carbomethyl)-L-lysine, (+/−) abscisic acid, sodium gluconate, monosodium glutamate, sodium acetate, homoeriodictyol, sterubin, eriodictyol, 2,4, dihydrobenzoic acid, neodiosmin, 1-carboxymethyl-5-hydroxy-2-hydroxymethylpyridinium, flavan-3-spiro-C-glycosides, poly-γ-glutamic acid, α,α-trehalose, taurine, (2)-gingerdione, 2,4,-dihydroxy benzoic acid, L-theanine, enterodiol, lariciresinol, enterolactone, matairesinol, and any combinations thereof.

In some further embodiments, the ingestible composition comprises one or more sour taste modulating compounds.

In some further embodiments, the ingestible composition comprises one or more mouthfeel modifying compounds. Such mouthfeel modifying compounds include, but are not limited to, tannins, cellulosic materials, bamboo powder, and the like.

In some further embodiments, the ingestible composition comprises one or more flavor masking compounds. Such flavor masking compounds include, but are not limited to, cellulosic materials, materials extracted from fungus, materials extracted from plants, citric acid, carbonic acid (or carbonates), and the like.

In some embodiments, the beverage product comprises carbon dioxide, for example, to form a carbonated beverage comprising a distilled spirits. Hard seltsers are a non-limiting example of such beverages.

### EXAMPLES

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

### Example 1

### Grain Neutral Spirit

Sensory testing was performed comparing two samples of unaged grain neutral spirit (20% alcohol by volume), where one sample contained no vanillyl ether compounds and the other sample contained 4-(ethoxy methyl)-2-methoxyphenol (vanillyl ethyl ether or VEE) at a concentration of 5 ppm. The taste testers were asked to describe the differences between the two samples on a qualitative basis. The results are summarized in Table 2.

TABLE 2

| | |
|---|---|
| Smell | Taste testers identified either no difference or identified the test |
| | sample (containing VEE) as having a slight rum-like smell relative |
| | to the control sample. |
| Burning/Heat | All taste testers identified the test sample as imparting less of a |
| | burning/hot sensation relative to the control sample. |
| Bitterness | All taste testers identified the test sample as having a less bitter |
| | taste. |
| Sweetness | All taste testers identified the test sample as having a slightly |
| | sweeter taste. |
| Smoothness/Balance/ | All taste testers identified the test sample as having greater |
| Roundness | smoothness, balance, and roundness; all taste testers identified the |
| | test sample as having a taste more characteristic of an aged spirit. |
| Overall Taste | All taste testers identified the test sample as having a better overall |
| | taste relative to the control sample. |

### Example 2

### Grain Neutral Spirit

Sensory testing was performed comparing two samples of unaged grain neutral spirit (20% alcohol by volume), where one sample contained no vanillyl ether compounds and the other sample contained 4-(ethoxymethyl)-2-methoxyphenol (vanillyl ethyl ether or VEE) at a concentration of 25 ppm. The taste testers were asked to describe the differences between the two samples on a qualitative basis. The results are summarized in Table 3.

TABLE 3

| | |
|---|---|
| Smell | Taste testers identified either no difference or identified the test |
| | sample (containing VEE) as having no smell but reduces the |
| | ethanol pungent smell |
| Tingling/Heat | All taste testers identified the test sample as imparting slightly |
| | increased tingling/hot perception very similar to ethanol profile |
| | while significantly increasing the alcohol perception; taste like |
| | higher alcohol spirit smooth finish. |
| Bitterness | All taste testers identified the test sample as having a less bitter |
| | taste. |
| Sweetness | All taste testers identified the test sample as having a slightly |
| | sweeter taste. |
| Smoothness/Balance/ | All taste testers identified the test sample as having greater |
| Roundness | smoothness (less harsh), balance, and roundness; all taste testers |
| | identified the test sample as having a taste more characteristic of |
| | an aged spirit. |
| Overall Taste | All taste testers identified the test sample as having a better |
| | overall taste relative to the control sample. |

1. A method of modifying a flavor of a distilled spirit, the method comprising introducing to the distilled spirit a vanillic ether compound, wherein the vanillyl ether compound is a compound of formula (I): 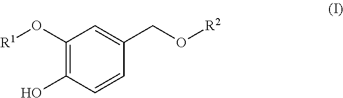
wherein:
R¹ is methyl or ethyl; and
R² is C₁₋₈ alkyl or C₃₋₈ cycloalkyl; or
R² is —CH₂-(phenyl), wherein the phenyl moiety is optionally substituted one or two times by substituents selected independently from the group consisting of —OH and —OCH₃.

2. The method of claim 1, wherein R¹ is methyl.

3. The method of claim 1, wherein R² is C₁₋₈ alkyl.

4. The method of claim 3, wherein R² is ethyl or butyl.

5. The method of claim 1, wherein the distilled spirit comprises at least 20% ethanol by volume.

6. The method uof claim 1, wherein the concentration of the vanillic ether compound ranges from 1 ppm to 100 ppm, based on the total weight of the distilled spirit.

7. The method of claim 1, wherein the distilled spirit comprises congeners.

8. A beverage composition, which comprises a distilled spirit and a vanillic ether compound, wherein the vanillyl ether compound is a compound of formula (I): 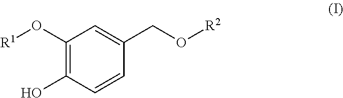
wherein:
R¹ is methyl or ethyl; and
R² is C₁₋₈ alkyl or C₃₋₈ cycloalkyl;
R² is —CH₂-(phenyl), wherein the phenyl moiety is optionally substituted one or two times by substituents selected independently from the group consisting of —OH and —OCH₃.

9. The beverage composition of claim 8, wherein R¹ is methyl.

10. The beverage composition of claim 8, wherein R² is C₁₋₈ alkyl.

11. The beverage composition of claim 10, wherein R² is ethyl or butyl.

12. The beverage composition of claim 8, wherein the distilled spirit comprises at least 20% ethanol by volume.

13. The beverage composition of claim 8, wherein the concentration of the vanillic ether compound ranges from 1 ppm to 100 ppm, based on the total weight of the beverage composition.

14. The beverage composition of claim 8, wherein the distilled spirit comprises congeners.
